(19) United States
(12) Patent Application Publication
(10) Pub. No.: US 2006/0241298 A1
(43) Pub. Date: Oct. 26, 2006

(54) Methods for synthesis of dicarbamate compounds and intermediates in the formation thereof

(76) Inventors: Henry Mortko, Bridgewater, NJ (US); Weixuan He, Monmouth Junction, NJ (US); Marc W. Andersen, Raleigh, NC (US); Anthony K. Dotse, Cary, NC (US); Jie Li, Cary, NC (US)

Correspondence Address: STERNE, KESSLER, GOLDSTEIN & FOX PLLC, 1100 NEW YORK AVENUE, N.W., WASHINGTON, DC, 20005, US

(21) Appl. No.: 11/407,909

(22) Filed: Apr. 21, 2006

Related U.S. Application Data
(60) Provisional application No. 60/673,361, filed on Apr. 21, 2005.

(51) Int. Cl. C07D 263/52; C07D 239/02; C07D 233/61; C07D 213/55

(52) U.S. Cl. 544/335; 544/336; 546/341; 548/200; 548/335.5; 548/216

(57) ABSTRACT

Disclosed is a method of making 2-substituted-2-halo-1,3-propanediols via reduction of corresponding malonate compounds. Also disclosed is a method of making 2-substituted-2-halo-1,3-dicarbamate compounds (such as halo derivatives of felbamate, including fluorofelbamate) via reduction of malonate compounds, followed by carbamoylation. Reduction of the malonate compounds is carried out using an electrophilic hydride reagent.

### BACKGROUND OF THE INVENTION

1. Field of the Invention

The present invention relates generally to processes for the preparation of dicarbamate compounds from diols and to the preparation of diol intermediates. In particular, the present invention provides processes for the production of dicarbamate compounds such as felbamate derivatives, including fluorofelbamate. Compounds provided by the synthetic methods of the present invention are useful in treating, ameliorating or preventing a variety of disorders, e.g., epilepsy.

2. Related Art

Felbamate is a known pharmaceutical compound (see U.S. Pat. Nos. 2,884,444 and 4,868,327, which are incorporated herein by reference in their entireties) that has been used successfully in controlling the seizures of epilepsy, a paroxysmal, self-sustaining and self-limited cerebral dysrhythmia that may be genetic or acquired in origin (see U.S. Pat. Nos. 4,978,680, 5,082,861 and 5,292,772, which are incorporated herein by reference in their entireties). Anti-epileptic drugs are thought to prevent or control seizures by acting on pathologically altered neurons or normal cells having restricted vascular supply, or an injured area in which the neurons of a nerve net have been destroyed.

Currently, drugs used in the treatment of epilepsy function as prophylactics against the symptoms of epilepsy, i.e., they act to reduce and control epileptic seizures as opposed to being curatives. The best anti-epileptic drugs have been characterized as non-toxic, non-sedative, long-acting and highly effective. One such drug is 2-phenyl-1,3-propanediol dicarbamate (I), known as felbamate.

However, the use of felbamate is limited due to the severity and frequency of occurrence of adverse reactions, notably aplastic anemia and hepatotoxicity. The toxicity of felbamate therapy is thought to be attributed to the metabolic formation of 2-phenylpropenal (commonly known as atropaldehyde) from felbamate. Felbamate derivatives, in particular 2-fluoro-2-phenyl-1,3-propanediol dicarbamate (II), known as fluorofelbamate (see U.S. Pat. No. 3,051,744, which is incorporated herein by reference in its entirety), can be substituted for felbamate in certain therapeutic uses that have been proposed for felbamate. Such therapeutic uses include, for example, treating or ameliorating neurological disorders, including, but not limited to, epileptic seizures, acute and chronic neurodegenerative conditions, neuropsychiatric disorders and pain; and treating, ameliorating or preventing tissue damage resulting from hypoxic conditions, including, but not limited to, cellular damage caused by myocardial or cerebral ischemic events (See U.S. Pat. Nos. 6,538,024 B1, 6,599,935 B2 and 6,759,402 B2, which are incorporated herein by reference in their entireties; and PCT Appl. Publ. No. WO 02/056827 A2). Moreover, these felbamate derivatives are reported to exhibit biological activity similar to felbamate but without the adverse reactions associated therewith (See id.). The improved toxicity profile of felbamate derivatives apparently is a result of the difference in metabolic processing of such derivatives versus felbamate. Specifically, the putative toxic chemical atropaldehyde is apparently prevented from forming in vivo when the hydrogen atom at the 2-position of felbamate is replaced with a halogen atom, such as fluorine.

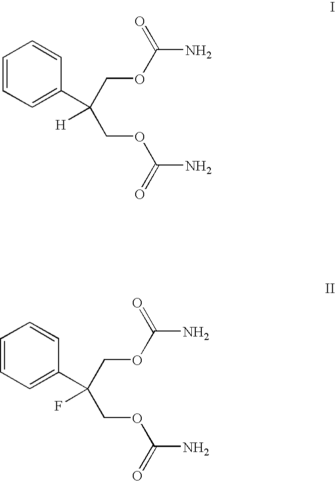

Fluorofelbamate can be prepared by methods known in the art by reduction of fluorinated malonate esters (III) using nucleophilic hydride reagents such as lithium aluminum hydride or sodium hydride as outlined below: 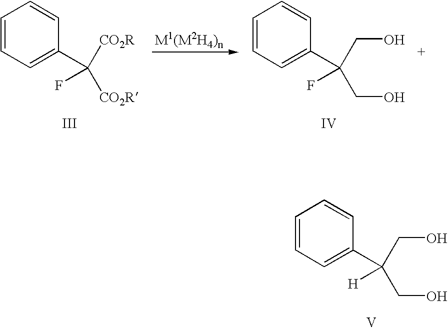
wherein R and R′ are alkyl groups; M¹ is an ion of a metal such as Na, K, Li or Ca; M² is an ion of B or Al; and n is 1 or 2, depending on the identity of M¹. Such synthetic approaches, however, give rise to side reactions that can affect the yield and purity of the final fluorofelbamate product.

A known side reaction that occurs when nucleophilic hydride reagents are used is defluorination, giving rise to compound V (and, consequently, lowering the yield of the desired F-Diol (IV)). For example, reduction with LiAlH₄ typically results in formation of the defluorinated product in the range of 10-12% (HPLC area under curve). This defluorinated material is difficult to remove by conventional means such as direct crystallization, distillation or simple chromatography. Ultimately this defluorination side reaction gives rise to felbamate as an impurity in the final fluorofelbamate product, an impurity that is not easily or inexpensively removed.

Thus, a need continues to exist for methods of making felbamate-derived compounds. Ideally, such methods would generally result in less dehalogenation than typically occurs with art-known methods.

### BRIEF SUMMARY OF THE INVENTION

In one aspect, the present invention is directed to methods of making 2-substituted-2-halo-1,3-propanediols via reduction of corresponding malonate compounds.

In another aspect, the present invention is directed to methods of making 2-substituted-2-halo-1,3-dicarbamate compounds, such as fluorofelbamate (II), via reduction of malonate compounds followed by carbamoylation.

Reduction of the malonate compounds is carried out using an electrophilic hydride reagent.

In one aspect, the present invention is directed to methods of making compounds of Formula VI: 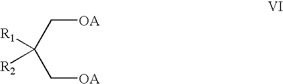
by reacting a compound of Formula VII: 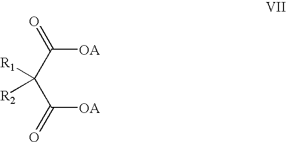
with an electrophilic hydride, wherein:

each occurrence of A is a cation;

R₂ is halo; and

R₁ is C₁₋₉ alkyl; C₃₋₉ cycloalkyl, optionally substituted once with C₁₋₉ alkyl; —(CH₂)ₘ-Het, wherein:
Het is a 5- or 6-membered heteroaryl group, optionally substituted with one or more substituents independently selected from halo, C₁₋₉ alkyl, halo(C₁₋₉)alkyl, hydroxyl, hydroxy(C₁₋₉)alkyl, C₁₋₉ alkoxy and NR₄R₅, wherein R₄ and R₅ are independently hydrogen or C₁₋₉ alkyl; and
m is 0, 1, 2, or 3;

or R₁ is

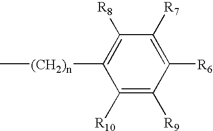

wherein:
n is 0, 1, 2 or 3; and
R₆, R₇, R₈, R₉and R₁₀ are independently selected from the group consisting of hydrogen, halo, C₁₋₉ alkyl, halo(C₁₋₉)alkyl, hydroxyl, hydroxy(C₁₋₉)alkyl, C₁₋₉ alkoxy and NR₄R₅, wherein R₄ and R₅ are independently selected from the group consisting of hydrogen and C₁₋₉ alkyl.

In another aspect, the present invention is directed to methods of converting the described compounds of Formula VII into compounds of Formula VIII:

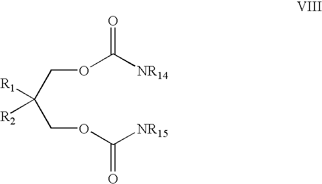

wherein R₁ and R₂ are described above; and

R₁₄ and R₁₅ are independently selected from the group consisting of hydrogen and C₁₋₄ alkyl.

Other embodiments of the present invention will be apparent to one of ordinary skill in the relevant arts in light of the following description of the invention, and in light of the claims.

### DETAILED DESCRIPTION OF THE INVENTION

The present invention provides processes for the preparation of dicarbamate compounds from diols and for the preparation of diol intermediates. In particular, the present invention provides processes for the production of dicarbamate compounds such as felbamate derivatives, including fluorofelbamate. Compounds provided by the synthetic methods of the present invention are useful in treating, ameliorating or preventing a variety of disorders, e.g., epilepsy.

In one aspect, the present invention is directed to methods of making compounds of Formula VI: 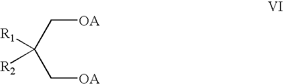
by reacting a compound of Formula VII: 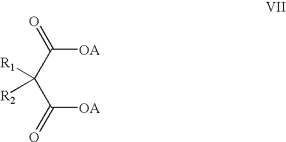
with an electrophilic hydride, wherein:

each occurrence of A is a cation;

R₂ is halo; and

R₁ is C₁₋₉ alkyl; C₃₋₉ cycloalkyl, optionally substituted once with C₁₋₉ alkyl; —(CH₂)ₘ-Het, wherein:
Het is a 5- or 6-membered heteroaryl group, optionally substituted with one or more substituents independently selected from halo, C₁₋₉ alkyl, halo(C₁₋₉)alkyl, hydroxyl, hydroxy(C₁₋₉)alkyl, C₁₋₉ alkoxy and NR₄R₅, wherein R₄ and R₅ are independently hydrogen or C₁₋₉ alkyl; and
m is 0, 1, 2, or 3;

or R₁ is

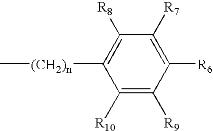

wherein:
n is 0, 1, 2 or 3; and
R₆, R₇, R₈, R₉ and R₁₀ are independently selected from the group consisting of hydrogen, halo, C₁₋₉ alkyl, halo(C₁₋₉)alkyl, hydroxyl, hydroxy(C₁₋₉)alkyl, C₁₋₉ alkoxy and NR₄R₅, wherein R₄ and R₅ are independently selected from the group consisting of hydrogen and C₁₋₉ alkyl.

Electrophilic hydrides useful in the methods of the present invention include, but are not limited to, compounds of formula BHRR′ and AlHRR′, wherein R and R′ independently represent hydrogen, C₁₋₆ alkyl or C₅₋₆ cycloalkyl. Useful electrophilic hydrides include BH₃ (“borane” or “diborane”), AlH₃ (“aluminum hydride”), ((CH₃)₂CH(CH₃)CH)₂BH, ((CH₃)₂CH(CH₃)CH)₂AlH, and the like, as well as catecholborane, bis(2,4,6-trimethylphenyl)borane, borabicyclo[3.3.1]nonane (9-BBN), trimethylamine-carbomethoxyborane and the like. More useful electrophilic hydrides include diborane and aluminum hydride, particularly diborane.

Any suitable borane complex may be used in the methods of the present invention. Useful borane complexes include, but are not limited to, BH₃.THF, BH₃.OEt₂, BH₃ .SMe₂, borane-1,2-bis(tert-butylthio)ethane, borane-ammonia, borane-t-butylamine, borane-N-ethyl-N-isopropylaniline, borane-N,N-diethylaniline, borane-N,N-diisopropylethylamine, BH₃.NHEt₂, BH₃ .NHMe₂, borane-diphenylphosphine, borane-isoamylsulfide, borane-1,4-oxathiane, borane-4-ethylmorpholine, borane-4-methylmorpholine, borane-morpholine, borane-pyridine, BH₃.NEt₃, borane-tributylphosphine, borane-triphenylphosphine and the like. More useful borane complexes include BH₃.THF.

Cations useful as A in the methods of the present invention include, but are not limited to, Group IA, Group IIA and Group IIIA cations such as H⁺, Li⁺, Na⁺, K⁺, Cs⁺, Mg²⁺, Ca²⁺, Sr²⁺, Ba²⁺, B³⁺, Al³⁺ and the like. Also useful are transition metal ions such as Co²⁺, Cu²⁺, Sc²⁺, Ni²⁺, Zn²⁺ and the like. More useful cations include H⁺, Li⁺, Na⁺, K⁺, Ca²⁺, Zn²⁺ and Al³⁺, particularly H⁺, Na⁺, K⁺ and Ca²⁺.

Each occurrence of A in the same instance of Formula VII is independent of the other. In addition, when A is a monovalent cation (e.g., H⁺, Na⁺ or K⁺), the two occurrences of A in the same instance of Formula VII may be the same or different. For example, each A may represent a H⁺ ion, or one A may represent a H⁺ ion while the other represents a Na⁺ ion. When A is a divalent cation (e.g., Ca²⁺), the two occurrences of A in the same instance of Formula VII may be the same or different, or both occurrences of A together may represent the same single ion. For example, each A may represent a Ca²⁺ ion, or both A's together may represent a Ca²⁺ ion. Combinations of cations with different valences (i.e., monovalent and/or divalent and/or trivalent) are also included. For example, one A may represent a Na⁺ ion while the other represents a Ca²⁺ ion, etc.

The preceding paragraph regarding each occurrence of A in the same instance of Formula VII is equally applicable to each occurrence of A in the same instance of Formula VI. Furthermore, the identity of A in the compound of Formula VI need not be the same as the identity of A in the compound of Formula VII.

Examples of 5- and 6-membered heteroaryl groups that are useful in accordance with the present invention include, but are not limited to, pyrrolyl, furanyl, thienyl, pyrazolyl, imidazolyl, oxazolyl, isoxazolyl, thiazolyl, isothiazolyl, triazolyl, oxadiazolyl, thiadiazolyl, tetrazolyl, oxatriazolyl, thiatriazolyl, pyridinyl, pyridazinyl, pyrimidinyl, pyrazinyl, triazinyl and the like. Each of these groups optionally can be substituted as described above.

More useful 5- and 6-membered heteroaryl groups include those attached via a ring carbon atom. Examples include, but are not limited to, 2-furanyl, 3-furanyl, 2-thienyl, 3-thienyl, 2-imidazolyl, 4-imidazolyl, 2-oxazolyl, 4-oxazolyl, 5-oxazolyl, 2-thiazolyl, 4-thiazolyl, 5-thiazolyl, 2-pyridinyl, 3-pyridinyl, 4-pyridinyl, 2-pyrimidinyl, 4-pyrimidinyl, 5-pyrimidinyl and 2-pyrazinyl. Other examples include, but are not limited to, 2-pyrrolyl, 3-pyrrolyl, 3-pyridazinyl and 4-pyridazinyl.

More useful substituted 5- and 6-membered heteroaryl groups include those attached via a ring carbon atom in which the substituent is attached to a ring carbon atom. Examples include, but are not limited to, 3-methylfuran-2-yl, 2-hydroxyfuran-3-yl, 5-bromothien-2-yl, 2-ethylthien-3-yl, 4-chloroimidazol-2-yl, 2-(trifluoromethyl)imidazol-4-yl, 5-isopropyloxazol-2-yl, 2-(fluoromethyl)oxazol-4-yl, 2-butyloxazol-5-yl, 4-iodothiazol-2-yl, 5-methylthiazol-4-yl, 2-hydroxythiazol-5-yl, 3-chloropyridin-2-yl, 4-(2,2,2-trifluoroethyl)pyridin-3-yl, 2-hydroxypyridin-4-yl, 4-isobutylpyrimidin-2-yl, 2-methylpyrimidin-4-yl, 2-chloropyrimidin-5-yl and 3-ethylpyrazin-2-yl. Other examples include, but are not limited to, 4-hydroxypyrrol-2-yl, 2-ethylpyrrol-3-yl, 4-(trifluoromethyl)pyridazin-3-yl and 6-fluoropyridazin-4-yl.

Examples of alkyl substituents useful in accordance with the present invention include, but are not limited to, C₁₋₆ alkyl, particularly C₁₋₄ alkyl. Examples of C₁₋₄ alkyl include methyl, ethyl, n-propyl, isopropyl, n-butyl, isobutyl, s-butyl and t-butyl. Examples of C₁₋₆ alkyl include, but are not limited to, 1-pentyl, 2-pentyl, 3-pentyl, isopentyl, neopentyl, 1-hexyl, 2-hexyl, 3-hexyl and isohexyl, as well as those listed for C₁₋₄ alkyl.

Examples of haloalkyl substituents useful in accordance with the present invention include, but are not limited to, halo(C₁₋₆)alkyl, particularly halo(C₁₋₄)alkyl.

Examples of hydroxyalkyl substituents useful in accordance with the present invention include, but are not limited to, hydroxy(C₁₋₆)alkyl, particularly hydroxy(C₁₋₄)alkyl.

Examples of alkoxy substituents useful in accordance with the present invention include, but are not limited to, C₁₋₆ alkoxy, particularly C₁₋₄ alkoxy.

Examples of cycloalkyl substituents useful in accordance with the present invention include, but are not limited to, C₃₋₆ cycloalkyl, particularly C₅₋₆ cycloalkyl. Examples of C₅₋₆ cycloalkyl include cyclopentyl and cyclohexyl. Examples of C₃₋₆ cycloalkyl include cyclopropyl and cyclobutyl, as well as those listed for C₅₋₆ cycloalkyl.

In certain embodiments R₂ is chloro or fluoro, particularly fluoro.

Suitable solvents in which the reaction may take place include, but are not limited to, tetrahydrofuran (THF), ether, benzene, toluene, xylene and the like, and mixtures thereof. More useful solvents include THF.

Suitable temperature ranges within which the reaction may take place include from about −10° C. to about 50° C. More useful temperature ranges within which the reaction may take place include from about 0° C. to about 25° C.

In one embodiment, the compounds made by the present invention are those of Formula VI:

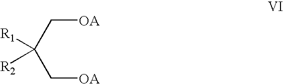

wherein:
A and R₂ are defined as above; and
R₁ is C₁₋₉ alkyl; C₃₋₉ cycloalkyl, optionally substituted once with C₁₋₉ alkyl;

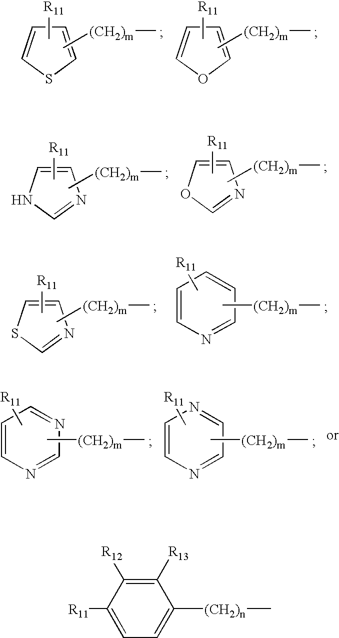

wherein:
m is 0, 1, 2 or 3;
n is 0, 1, 2 or 3; and
R₁₁, R₁₂ and R₁₃ are independently selected from the group consisting of hydrogen, halo, C₁₋₄ alkyl, halo(C₁₋₄)alkyl and hydroxyl.

In this embodiment, useful R₂ include fluoro and chloro, particularly fluoro.

In this embodiment, useful R₁₁, R₁₂ and R₁₃ include hydrogen.

One group of useful compounds in this embodiment includes those wherein R₂ is fluoro; m is 0; and one of R₁₁, R₁₂ or R₁₃ is hydrogen, halo, C₁₋₄ alkyl, halo(C₁₋₄)alkyl or hydroxyl, and the other two are hydrogen; particularly wherein R₁₁, R₁₂ and R₁₃ are each hydrogen.

One group of useful compounds in this embodiment includes those wherein m is 0; and n is 0.

One group of useful compounds in this embodiment includes those wherein:

R₂ is fluoro; and

R₁ is C₃₋₉ cycloalkyl,

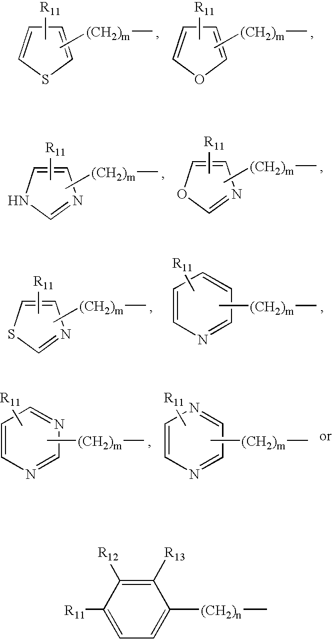

wherein:
m is 0;
n is 0; and
R₁₁, R₁₂ and R₁₃ are each hydrogen.

In one embodiment, the compounds made by the methods of the present invention are those of Formula VI: 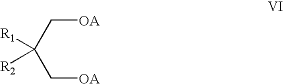
wherein:

A and R₂ are defined as above; and

R₁ is

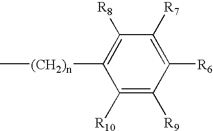

wherein:
n is 0; and
R₆, R₇, R₈, R₉ and R₁₀ are independently selected from the group consisting of hydrogen, halo, C₁₋₄ alkyl, halo(C₁₋₄)alkyl and hydroxyl.

In this embodiment, useful R₂ include fluoro and chloro, particularly fluoro.

One group of useful compounds in this embodiment includes those wherein R₈, R₉ and R₁₀ are each hydrogen.

One group of useful compounds in this embodiment includes those wherein R₇, R₉ and R₁₀ are each hydrogen.

One group of useful compounds in this embodiment includes those wherein R₇, R₈, R₉ and R₁₀ are each hydrogen. More useful are compounds wherein R₇, R₈, R₉ and R₁₀ are each hydrogen, and R₂ is fluoro.

One group of useful compounds in this embodiment includes those wherein R₆, R₇, R₈, R₉ and R₁₀ are each hydrogen, i.e., R₁ is phenyl. More useful are compounds wherein R₁ is phenyl and R₂ is fluoro.

In another aspect, the present invention is directed to methods of converting the described compounds of Formula VII into compounds of Formula VIII:

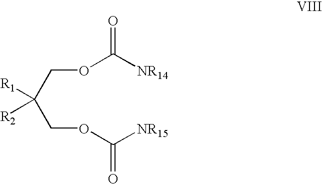

wherein R₁ and R₂ are as described above; and
R₁₄ and R₁₅ are independently selected from the group consisting of hydrogen and C₁₋₄ alkyl. In particularly preferred embodiments, R₁₄ or R₁₅, or both R₁₄ and R₁₅, are hydrogen.

Particularly preferred compounds produced by the methods of the present invention include derivatives of felbamate (I), including fluorofelbamate (II) and other halo-substituted felbamate derivatives.

Methods of effecting the conversion are known in the art, and any suitable method may be employed. For example, treating a compound of Formula VII with a source of ammonia and a coupling agent affords a compound of Formula VIII wherein R₁₄ and R₁₅ are each hydrogen. Suitable sources of ammonia include, but are not limited to, ammonia and compounds capable of providing ammonia in situ, e.g., ammonium carbonate. Suitable coupling agents include, but are not limited to, 1,1′-carbonyldiimidazole (CDI). Methods useful to effect the conversion include, but are not limited to, treatment with CDI and ammonium carbonate, particularly in the presence of molecular sieves; treatment with CDI and liquid ammonia; and treatment with phosgene and NH₄OH. More useful methods include treatment with CDI and ammonium carbonate, particularly in the presence of molecular sieves.

Some of the compounds described herein may contain one or more asymmetric centers and thus give rise to enantiomers, diastereomers, and other stereoisomeric forms. The present invention is also meant to encompass the production of all such possible forms as well as their racemic and resolved forms and mixtures thereof. When the compounds described herein contain olefinic double bonds or other centers of geometric asymmetry, and unless specified otherwise, it is intended to include both E and Z geometric isomers. All tautomers, and methods of their production, are intended to be encompassed by the present invention as well.

The compounds produced by the methods of the present invention are suitable for use in treating, ameliorating and/or preventing a variety of neurological disorders or conditions, including, but not limited to, epileptic seizures, acute and chronic neurodegenerative conditions, neuropsychiatric disorders and pain; and treating, ameliorating or preventing tissue damage resulting from hypoxic conditions, including, but not limited to, cellular damage caused by myocardial or cerebral ischemic events. (See published PCT Appl. Publ. No. WO 02/056827 A2). Thus, in another aspect, the present invention provides compounds produced by the methods of the present invention, and pharmaceutical compositions comprising such compounds and one or more pharmaceutically acceptable carriers or excipients therefor. Suitable pharmaceutically acceptable carriers or excipients that can be used in accordance with the present invention will be familiar to those of ordinary skill in the art.

When any variable occurs more than one time in any constituent or formula, its definition at each occurrence is independent of its definition at every other occurrence. Also, combinations of substituents and/or variables are permissible only if such combinations result in stable compounds.

### DEFINITIONS

The term “alkyl” as employed herein by itself or as part of another group refers to both straight and branched chain radicals of up to 10 carbons, unless the chain length is otherwise limited, such as methyl, ethyl, propyl, isopropyl, butyl, t-butyl, pentyl, hexyl, isohexyl, heptyl, 4,4-dimethylpentyl, octyl, 2,2,4-trimethylpentyl, nonyl and decyl.

The term “halogen” or “halo” as employed herein by itself or as part of another group refers to fluoro, chloro, bromo or iodo.

The term “haloalkyl” as employed herein refers to alkyl groups wherein one or more hydrogens thereof are substituted by one or more halo moieties. Typical examples include fluoromethyl, difluoromethyl, trifluoromethyl, trichloroethyl, trifluoroethyl, fluoropropyl, and bromobutyl.

The term “cycloalkyl” as employed herein by itself or as part of another group refers to cycloalkyl groups containing 3 to 9 carbon atoms. Typical examples include cyclopropyl, cyclobutyl, cyclopentyl, cyclohexyl, cycloheptyl, cyclooctyl and cyclononyl.

The term “heteroaryl” as employed herein refers to groups having 5 to 14 ring atoms; 6, 10 or 14 pi electrons shared in a cyclic array; and containing carbon atoms and 1, 2, 3, or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4αH-carbazozolyl, carbozolyl, β-carbolinyl, phenanthridinyl, acridinyl, perimidinyl, phenanthrolinyl, phenazinyl, isothiazolyl, phenothiazinyl, isoxazolyl, furazanyl, phenoxazinyl and tetrazolyl groups).

The terms “hydroxy” and “hydroxyl” are used interchangeably herein to refer to the radical —OH.

The term “hydroxyalkyl” as employed herein refers to alkyl groups wherein one or more hydrogens thereof are substituted by one or more hydroxyl moieties.

The terms “alkoxy”, “alkyloxy” and “alkoxyl” are used interchangeably herein to refer to the radical —OR, where R is alkyl. Typical examples include methoxy, ethoxy, isopropyloxy, sec-butyloxy, and t-butyloxy.

A ring structure having one or more bonds extending from the center of the ring indicates that the point of attachment may be to any of the carbon atoms of the ring. For example, the structure: 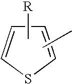
indicates that the thienyl group may be attached via any of its ring carbon atoms, and that the R substituent is attached to the thienyl group at one of the remaining ring carbon atoms.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached, or a sulfur atom to which three different groups are attached, where the sulfur atom and its attached groups form a sulfoxide, sulfinic ester, sulfonium salt or sulfite.

The term “enantiomer” or “enantiomeric” refers to a molecule that is nonsuperimposable on its mirror image and hence optically active wherein the enantiomer rotates the plane of polarized light in one direction and its mirror image rotates the plane of polarized light in the opposite direction.

The term “racemic” refers to a mixture of equal parts of enantiomers and which is optically inactive.

The term “resolution” refers to the separation or concentration or depletion of one of the two enantiomeric forms of a molecule. The phrase “enantiomeric excess” refers to a mixture wherein one enantiomer is present in a greater concentration than its mirror image molecule.

As used herein, the terms “about” or “approximately” when referring to any numerical value are intended to mean a value of ±10% of the stated value. For example, “about 50° C.” (or “approximately 50° C.”) encompasses a range of temperatures from 45° C. to 55° C., inclusive. Similarly, “about 100 mM” (or “approximately 100 mM”) encompasses a range of concentrations from 90 mM to 110 mM, inclusive.

Having now described the present invention in detail, the same will be more clearly understood by reference to the following examples, which are included herewith for purposes of illustration only and are not intended to be limiting of the invention.

### EXAMPLES

In the following examples, the term “parts” refers to weight/weight when a solid is used, and volume/volume when a liquid is used. Amounts of defluorinated products and other side products were determined by HPLC and are reported as % AUC (area under curve).

### Example 1

### 2-Fluoro-2-phenyl-malonic acid diethyl ester (F—PMADE)

Under an atmosphere of nitrogen, 0.14 parts of 95% sodium hydride were placed in a reaction vessel. Tetrahydrofuran (THF) (4.21 parts) was carefully added, and the mixture was stirred and cooled externally with ice-water. Ethanol (0.03 parts) was added followed by slow addition of 1.00 part of 2-phenyl-malonic acid diethyl ester (PMADE) in 1.17 parts THF at a rate to maintain the temperature at −10° C. to 5° C. During this period a strong evolution of hydrogen gas was observed. After addition of the ester, the mixture was stirred for approximately 2 h at less than 5° C. Selectfluor® (1-(chloromethyl)-4-fluoro-1,4-diazoniabicyclo[2.2.2]octane bis(tetrafluoro-borate) (1.98 parts) was added in portions so as to maintain the temperature below 10° C. The mixture was allowed to slowly warm up and stir for 8 h to 18 h. A small amount of methanol (0.06 parts) was added to ensure the excess amount of sodium hydride was destroyed, the suspension was suction filtered, and the filter cake was washed with 2.69 parts of THF. The resulting filtrate was then suction-filtered again through 0.19 parts of silica gel 60 and concentrated in vacuo. The dark residue was dissolved in 2.15 parts methyl tertiary-butyl ether (MTBE) and washed with 2.15 parts of water followed by 1.08 parts of brine solution. The organic layer was then dried over 0.27 parts sodium sulfate, filtered, and concentrated to an oil that was used in the next step without further purification. NMR analysis of the crude product indicated the presence of ca. 4% PMADE starting material along with the monodecarboxylated fluorinated species 2-fluoro-2-phenyl acetic acid ethyl ester at 0.2% and unknown impurity at 5-9% respectively. The overall purity of F—PMADE was estimated at 87-91%.

F—PMADE: ¹H-NMR (d₆-DMSO, 500 MHz) 67 7.47 (m, 5 H, PhH), 4.34-4.25 (m, 4 H, CH₂), 1.22 (t, 6 H, CH₃). Partial ¹H-NMR data for 2-fluoro-2-phenyl acetic acid ethyl ester: 6.16, 6.06 (d, CHFPh), 4.22-4.12 (m, 4 H, CH₂); PMADE: 4.93 (s, 1H, CHPh) and unknown impurity: 4.22-4.12 (m, 2-4 H) and 4.10 (q, 2-4 H).

### Example 2

### 2-Fluoro-2-phenyl-malonic acid dipotassium Salt (F—K₂PMA)

An ice-cooled solution of 1.00 part of F—PMADE in 15.47 parts of ethanol was treated slowly with a solution of 0.77 parts of potassium hydroxide in 3.02 parts of ethanol so as to maintain a temperature of −10 to 10° C. During this addition the reaction became very thick and was stirred for an additional 2 h, and was then isolated by suction-filtration. The wet crude solid was then slurried in 3.23 parts of methanol for approximately 2 h and isolated by suction filtration. The solids were washed with 1.04 parts of methanol. The wet solids were then re-suspended in 3.23 parts of methanol, stirred for approximately 2 h, filtered, washed with 1.04 parts of methanol, and dried in vacuo at 30-40° C. The yield of F—K₂PMA is typically 65-72% of theory. ¹H-NMR (D₂O, 500 MHz) δ 7.40 (m). HPLC analysis indicated a purity of 99.91% AUC. Under the following HPLC conditions, the retention times were: K₂PMA (i.e., defluorinated) (15.5 min), F—K₂PMA (22.2 min).

| | |
|---|---|
| Column: | ES Industries FluoroSep-RP phenyl, |
| | 3 μm, 25 cm × 4.6 mm |
| Mobile phase: | CH₃CN/H₂O/TFA = 20/80/0.1 (v/v) |
| Flow rate: | 0.75 mL/min |
| Detector: | UV 210 nm |
| Injection volume: | 20 μL (nominal) |
| Column temperature: | 25 ± 1° C. |
| Run time: | 20 min |

### Example 3

### 2-Fluoro-2-phenyl-1,3-propanediol (F-Diol)

To 14.4 parts of a 1 M solution of diborane in THF solution (1.15 L, 4 eq) was added 1.00 parts of F—K₂PMA slowly at ambient temperature. Gas evolution was observed during the addition. The reaction mixture was stirred at ambient temperature overnight. The reaction mixture was then externally cooled with an ice bath (typically, the reaction mixture is kept at a temperature of from 2-5° C. for 12-6 h) and was carefully treated with 6.25 parts of methanol. Solvents were removed under reduced pressure to leave a white paste, which was treated with 3.12 parts of 10% aqueous HCl solution followed by 6.88 parts of water. This aqueous mixture was washed with hexane, then saturated with NH₄Cl, followed by extraction with ethyl acetate (EtOAc). The combined EtOAc solution was washed with brine, followed by saturated aqueous NaHCO₃ solution, brine, and dried over anhydrous MgSO₄. After removal of solvent, crude F-Diol was obtained as a pale yellow solid in 74% weight yield. NMR analysis was consistent with the structure of F-Diol with about 2-3% of defluorinated material (2-phenyl-1,3-propanediol (“Diol”)). Under the following HPLC conditions, the retention times were: F-Diol (5.8 min), Diol (6.2 min).

| | |
|---|---|
| Column: | Kromasil C4, 5 μm, 25 cm × 4.6 mm |
| Mobile phase: | THF/MeOH/H₂O = 3.5/20.0/76.5 (v/v) |
| Flow rate: | 1.5 mL/min (nominal) |
| Detector: | UV 210 nm |
| Injection volume: | 20 μL (nominal) |
| Column temperature: | 35 ± 1° C. |
| Run time: | 20 min |

### Example 4

### 2-Fluoro-2-phenyl-1,3-propanediol (F-Diol)

A flask was charged with 1.00 part of F—K₂PMA and 2.50 parts of THF. The thick slurry was externally cooled with ice and treated dropwise with a solution of 1.83 parts of 4 N HCl in dioxane so as to maintain the temperature between 2.5 and 10° C. After the addition was complete, the slurry was stirred for an additional 0.5 h and 14.59 parts of a 1 M solution of diborane in THF was added so as to maintain the temperature between 2.5° C. and 12° C. An initial exotherm was accompanied by evolution of gas. After the addition was complete, the cooling bath was removed and the mixture was stirred at ambient temperature for 18-24 h. The mixture was then externally cooled with ice and carefully treated with 2.50 parts of aqueous 1 N HCl, during which period an initial exotherm was observed accompanied by gas evolution. During the addition, the temperature climbed from −5° C. to 9° C. at which point 3.75 parts of water and 3.75 parts of ethyl acetate were added. The phases were vigorously mixed and separated. The aqueous phase was removed and extracted with 1.25 parts of ethyl acetate. The organic phases were combined and washed with 2.50 parts of brine. The organic phase was then washed with 2.50 parts of saturated aqueous sodium bicarbonate followed by 1.25 parts of brine. The organic layer was then dried over 0.6 parts of sodium sulfate, filtered, and concentrated in vacuo to a thick residue. The residue was then concentrated three times from 1.90 parts each of methanol. The resulting semi-solid material was then dissolved in 3.5 parts of hot toluene and concentrated while warming to 50-80° C., removing 2-3 parts of toluene. The resulting toluene solution was then filtered hot and allowed to crystallize with stirring for 18-48 h at ambient temperature, then 12-24 h at 0-4° C. The white crystalline material was isolated by suction filtration. After drying, the yield of 2-fluoro-2-phenyl-1,3-propanediol was 85-90% of theoretical. HPLC analysis typically shows >98% (AUC) F-Diol along with 0.5-1.1% defluorinated material (2-phenyl-1,3-propanediol (“Diol”)). ¹H-NMR (d₆-DMSO, 500 MHz) δ 7.40-7.20 (m, 5 H, PhH), 5.0 (t, 2 H, OH), 3.83-3.70 (m, 4 H, CH₂). Under the HPLC conditions described for Example 2, the retention times were: Diol (8.1 min), F-Diol (8.5 min).

### Example 5

### 2-Fluoro-2-phenyl-1,3-propanediol dicarbamate (Fluorofelbamate)

A flask was charged with 1.00 part of F-Diol and 9.50 parts of THF. The resulting solution was treated with 2.39 parts of 1,1′-carbonyldiiumidazole (CDI) in a single portion. After several hours a heavy precipitate formed which was stirred an additional 18-24 h. Next, 1.00 part of powdered activated molecular sieves (4 Å, 25μ) was added followed by 3.4 parts of ammonium carbonate. The slurry was stirred for 18-24 h, then treated with an additional 3.4 parts of ammonium carbonate. After an additional 18-24 h, the reaction mixture was allowed to settle for 2-24 h and the supernatant was removed. The remaining slurry was treated with ethyl acetate (5 parts), stirred, and filtered to remove solids. The filter cake was washed three times with 2.5 parts each of ethyl acetate. The organic phases were combined and concentrated to an oil, then dissolved in 5 parts ethyl acetate, and washed with 2.5 parts of water then 3 parts of 6 N hydrochloric acid. (An additional wash may be necessary if the pH of the aqueous acid wash is still basic by pH paper.) The ethyl acetate layer was then washed with 3 parts brine solution followed by 3 parts of sodium bicarbonate. The organic layer was dried over 1.0 part sodium sulfate, filtered, and concentrated in vacuo, while maintaining a bath temperature of 60-80° C., to a light-syrup (leaving approximately 1-2 parts ethyl acetate). This solution was then added to 5 parts of MTBE with stirring at which point crystallization commenced. The resulting white slurry was stirred 14-24 h and the solids were isolated by filtration and dried in vacuo at 60° C. The yield of crude 2-fluoro-2-phenyl-1,3-propanediol dicarbamate is typically 78-85% of theoretical. HPLC analysis indicated >98-99% (AUC) purity along with 0.5% 2-phenyl-1,3-propanediol and 0.3-0.5% 2-fluoro-2-phenyl-1,3-propanediol monocarbamate (“F-monocarbamate”). The crude product was further purified by dissolving 1.00 part fluorofelbamate in 10 parts of hot methanol-water (1:4). Cooling to ambient temperature and stirring overnight, followed by filtration, afforded the title compound as a white crystalline solid. Yields of crystallization processes are typically 93-97%. HPLC analysis indicated >99.5% AUC fluorofelbamate. Typically, less than 0.35% felbamate is present by HPLC. ¹H-NMR (d₆-DMSO, 500 MHz) 67 7.50-7.20 (m, 5 H, PhH), 6.8-6.2 (bd, 4 H, NH2), 4.42-4.20 (m, 4 H, CH₂). Under the HPLC conditions described for Example 3, the retention times were: F-Diol (5.8 min), Diol (6.2 min), monocarbamate (8.8 min), F-monocarbamate (9.3 min), felbamate (12.3 min), fluorofelbamate (15.8 min).

Having now fully described this invention, it will be understood by those of ordinary skill in the art that the same can be performed within a wide and equivalent range of conditions, formulations and other parameters without affecting the scope of the invention or any embodiment thereof.

What is claimed is:

1. A method of making a compound of Formula VI: 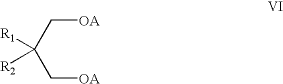
comprising reacting a compound of Formula VII: 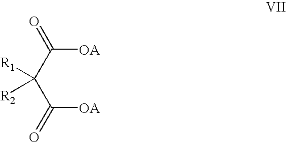
with an electrophilic hydride, wherein:
each occurrence of A is a cation;
R₂ is halo; and
R₁ is C₁₋₉ alkyl; C₃₋₉ cycloalkyl, optionally substituted once with C₁₋₉ alkyl; or —(CH₂)ₘ-Het, wherein:
Het is a 5- or 6-membered heteroaryl group, optionally substituted with one or more substituents independently selected from halo, C₁₋₉ alkyl, halo(C₁₋₉)alkyl, hydroxyl, hydroxy(C₁₋₉)alkyl, C₁₋₉ alkoxy and NR₄R₅, wherein R₄ and R₅ are independently hydrogen or C₁₋₉ alkyl; and
m is 0, 1, 2 or 3,
or R₁ is 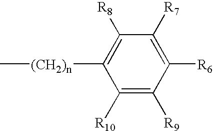
wherein:
n is 0, 1, 2 or 3; and
R₆, R₇, R₈, R₉ and R₁₀ are independently selected from the group consisting of hydrogen, halo, C₁₋₉ alkyl, halo(C₁₋₉)alkyl, hydroxyl, hydroxy(C₁₋₉)alkyl, C₁₋₉ alkoxy and NR₄R₅, wherein R₄ and R₅ are independently selected from the group consisting of hydrogen and C₁₋₉ alkyl.

2. The method according to claim 1, wherein:
R₁ is C₁₋₉ alkyl; C₃₋₉ cycloalkyl, optionally substituted once with C₁₋₉ alkyl; 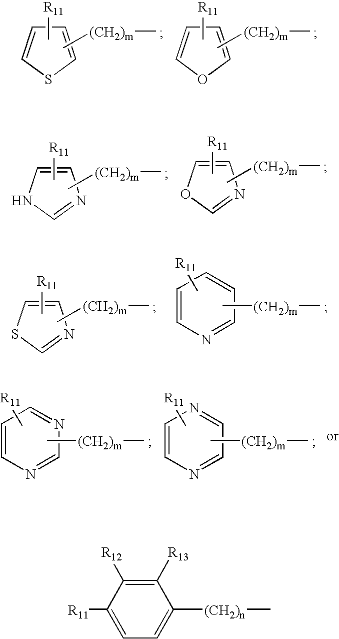
wherein:
m is 0, 1, 2 or 3;
n is 0, 1, 2 or 3; and
R₁₁, R₁₂ and R₁₃ are independently selected from the group consisting of hydrogen, halo, C₁₋₄ alkyl, halo(C₁₋₄)alkyl and hydroxyl.

3. The method according to claim 2, wherein R₂ is fluoro or chloro.

4. The method according to claim 2, wherein R₂ is fluoro.

5. The method according to claim 2, wherein R₁₁, R₁₂ and R₁₃ are each hydrogen.

6. The method according to claim 2, wherein:
R₂ is fluoro; and
R₁₁, R₁₂ and R₁₃ are each hydrogen.

7. The method according to claim 2, wherein:
R₂ is fluoro;
m is 0; and
one of R₁₁, R₁₂ or R₁₃ is hydrogen, halo, C₁₋₄ alkyl, halo(C₁₋₄)alkyl or hydroxyl, and the other two are hydrogen.

8. The method according to claim 7, wherein R₁₁, R₁₂ and R₁₃ are each hydrogen.

9. The method according to claim 2, wherein:
m is 0; and
n is 0.

10. The method according to claim 2, wherein:
R₂ is fluoro; and
R₁ is C₃₋₉ cycloalkyl, 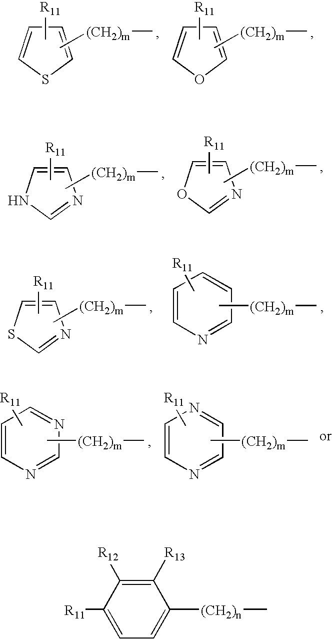
wherein:
m is 0;
n is 0; and
R₁₁, R₁₂ and R₁₃ are each hydrogen.

11. The method according to claim 1, wherein R₁ is 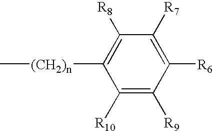
wherein:
n is 0; and
R₆, R₇, R₈, R₉ and R₁₀ are independently selected from the group consisting of hydrogen, halo, C₁₋₄ alkyl, halo(C₁₋₄)alkyl and hydroxyl.

12. The method according to claim 11, wherein R₂ is fluoro or chloro.

13. The method according to claim 11, wherein R₂ is fluoro.

14. The method according to claim 11, wherein R₈, R₉ and R₁₀ are each hydrogen.

15. The method according to claim 11, wherein R₇, R₉ and R₁₀ are each hydrogen.

16. The method according to claim 11, wherein R₇, R₈, R₉ and R₁₀ are each hydrogen.

17. The method according to claim 16, wherein R₂ is fluoro.

18. The method according to claim 1, wherein:
R₂ is fluoro; and
R₁ is phenyl.

19. The method according to claim 1, wherein the electrophilic hydride is catecholborane, bis(2,4,6-trimethylphenyl)borane, borabicyclo[3.3.1]nonane, trimethylamine-carbomethoxyborane, or a compound of formula BHRR′ or AlHRR′, wherein R and R′ independently represent hydrogen, C₁₋₆ alkyl or C₅₋₆ cycloalkyl;.

20. The method according to claim 1, wherein the electrophilic hydride is diborane, aluminum hydride, ((CH₃)₂CH(CH₃)CH)₂BH, ((CH₃)₂CH(CH₃)CH)₂AlH, catecholborane, bis(2,4,6-trimethylphenyl)borane, borabicyclo[3.3.1]nonane or trimethylamine-carbomethoxyborane.

21. The method according to claim 1, wherein the electrophilic hydride is diborane or aluminum hydride.

22. The method according to claim 1, wherein the electrophilic hydride is diborane.

23. The method according to claim 1, wherein each occurrence of A is independently selected from the group consisting of Group IA cations, Group IIA cations, Group IIIA cations, Co²⁺, Cu²⁺, Sc²⁺, Ni²⁺ and Zn²⁺.

24. The method according to claim 1, wherein each occurrence of A is independently selected from the group consisting of H⁺, Li⁺, Na⁺, K⁺, Cs⁺, Mg²⁺, Ca²⁺, Sr²⁺, Ba²⁺, B³⁺ and Al³⁺.

25. The method according to claim 1, wherein each occurrence of A is independently selected from the group consisting of H⁺, Li⁺, Na⁺, K⁺, Ca²⁺, Zn²⁺ and Al³⁺.

26. The method according to claim 1, wherein each occurrence of A is independently selected from the group consisting of H⁺, Na⁺, K⁺ and Ca²⁺.

27. The method according to claim 1, wherein said reacting takes place in a solvent selected from THF, ether, benzene, toluene, xylene and mixtures thereof.

28. The method according to claim 1, wherein said reacting takes place in THF.

29. The method according to claim 1, wherein said reacting takes place within a temperature range of from about −10° C. to about 50° C.

30. The method according to claim 1, wherein said reacting takes place within a temperature range of from about 0° C. to about 25° C.

31. The method according to any of claims 1-30, further comprising converting the compound of Formula VII into a compound of Formula VIII: 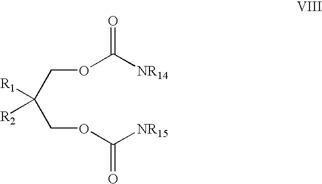
wherein:
R₁₄ and R₁₅ are independently selected from the group consisting of hydrogen and C₁₋₄ alkyl.

32. The method according to claim 31, wherein R₁₄ and R₁₅ are each hydrogen.

33. The method according to claim 31, wherein the compound of Formula VIII is fluorofelbamate.

34. The method according to claim 32, wherein said converting comprises treating the compound of Formula VII with a source of ammonia and a coupling agent.

35. The method according to claim 34, wherein the source of ammonia is ammonium carbonate and the coupling agent is 1,1′-carbonyl-diimidazole.

36. The method according to claim 34, wherein the source of ammonia is ammonium carbonate and the coupling agent is 1,1′-carbonyl-diimidazole, and wherein said converting takes place in the presence of molecular sieves.

37. The method according to claim 1, wherein:
the electrophilic hydride is diborane;
each occurrence of A is independently selected from the group consisting of H⁺, Na⁺, K⁺ and Ca²⁺;
R₂ is fluoro; and
R₁ is phenyl.

38. The method according to claim 37, wherein said reacting takes place in THF.

39. The method according to any of claims 37-38, further comprising converting the compound of Formula VII into a compound of Formula II:

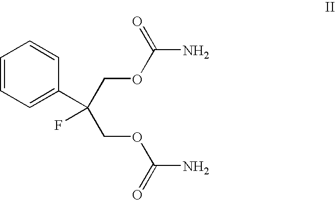

40. The method according to claim 39, wherein said converting comprises treating the compound of Formula VII with ammonium carbonate and 1,1′-carbonyldiimidazole.

41. The method according to claim 40, wherein said converting takes place in the presence of molecular sieves.
